(19) United States
(12) Patent Application Publication
(10) Pub. No.: US 2026/0146148 A1
(43) Pub. Date: May 28, 2026

(54) RUBBER COMPOSITION CAPABLE OF PROTECTING AGAINST METAL ION AGING AND USE THEREOF

(71) Applicant: SENNICS CO., LTD., Shanghai (CN)

(72) Inventors: Yang GAO, Shanghai (CN); Hao WANG, Shanghai (CN); Xiangyun GUO, Shanghai (CN)

(21) Appl. No.: 19/441,737

(22) Filed: Jan. 6, 2026

Related U.S. Application Data
(63) Continuation of application No. PCT/CN2024/094380, filed on May 21, 2024.

(30) Foreign Application Priority Data
Jul. 18, 2023 (CN) 202310885309.2

(51) Int. Cl. C08L 7/00; B60C 1/00; C08K 3/04; C08K 3/06; C08K 3/22; C08K 3/36; C08K 3/38; C08K 5/18; C08K 5/47

(52) U.S. Cl. CPC C08L 7/00; B60C 1/0041; C08K 3/04; C08K 3/06; C08K 3/36; C08K 5/18; C08K 5/47; C08K 2003/2296; C08K 2003/387; C08L 2201/08

(57) ABSTRACT

A compound of formula A which is used as an anti-aging agent, a rubber composition comprising the compound of formula A, and a method of using the compound in the rubber composition are provided. The rubber composition of the present invention is used as an entire or partial rubber matrix of a tire, especially as a tire belt rubber. The anti-aging agent of the present invention can provide good rubber-steel wire adhesion performance, improve the thermal oxidative aging resistance of the rubber composition, and especially improve the mechanical properties and adhesion properties after aging in the presence of metal ions, prolonging the service life of tires.

### CROSS-REFERENCE TO RELATED APPLICATIONS

The subject application is a continuation of PCT/CN2024/094380 filed on May 21, 2024, which in turn claims priority on Chinese Patent Application No. CN202310885309.2 filed on Jul. 18, 2023 in China. The contents and subject matters of the PCT international application and the Chinese priority application are incorporated herein by reference.

### TECHNICAL FIELD

The present invention belongs to the field of rubber science, in particular, relating to a rubber composition capable of protecting against metal ion aging and the use thereof.

### BACKGROUND ART

As one of the main components of an automobile, the tire plays an important role in bearing vehicle weight and transmitting traction and braking forces. As the tire industry continues to develop, meridian tires have been widely adopted where using the steel cords have become a very common reinforcement method. Therefore, the adhesion performance between the rubber and the steel wire has become crucial. Research has found that cobalt salts can significantly promote the rubber-steel cord adhesion. It is generally believed that cobalt salts enhance the activity of sulfur, increase the formation rate of copper sulfide at the adhesion interface, which is beneficial for improving adhesion performance.

Although the addition of cobalt salts can enhance rubber-steel cord adhesion performance, it detrimentally affects the aging performance of the rubber composition.

An unsaturated rubber is prone to react with oxygen. Research indicates that the oxidation of the unsaturated rubber occurs via a free radical chain reaction mechanism and has the characteristic of autocatalytic, while metal ions such as copper, manganese, and cobalt can play a catalytic role for the oxidation reaction of a rubber. Metal ion catalysis can greatly increase the oxidation rate, accelerate the thermal oxidation of the rubber, which is of great harm to the rubber.

In the existing technology, an antidegradant is mostly used to inhibit the catalytic effect of metal ions, thereby improving the thermal oxidative aging resistance and other properties of the rubber product. 6PPD is a major antidegradant that has been widely used in the rubber industry, which inhibits ozone aging and thermal oxidative aging of the rubber product and slows down rubber degradation under static conditions. However, 6PPD exhibits certain polluting properties, and its extensive use as a single component antidegradant in tire formulations increases production costs, while it cannot fully meet the requirements for performance retention under some specific aging conditions during the actual use of tires and is prone to cause issues such as blooming.

### SUMMARY OF THE INVENTION

To overcome the problems in the prior art, the present invention provides a rubber composition with excellent protection against metal ion aging and the use thereof. The rubber composition of the present invention is used as an entire or partial rubber matrix of a tire, especially as a tire belt rubber. The antidegradant of the present invention provides good rubber-steel wire adhesion performance, improves the thermal oxidative aging resistance and protection against metal ion in the rubber composition, and especially improves the mechanical performance and adhesion performance after aging in the presence of metal ions, prolonging the service life of tires.

Specifically, the present invention provides a compound of formula A, which is used as an antidegradant:

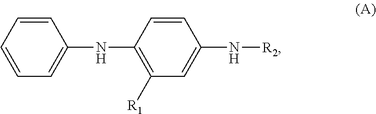

wherein R₁ is selected from C1-C8 alkyl, and R₂ is selected from C1-C8 alkyl in formula A.

In one or more embodiments of the present invention, R₁ is methyl, and R₂ is selected from C₃-C₆ alkyl in formula A.

In one or more embodiments of the present invention, the compound of formula A is a compound of formula I, a compound of formula II, or a compound of formula III:

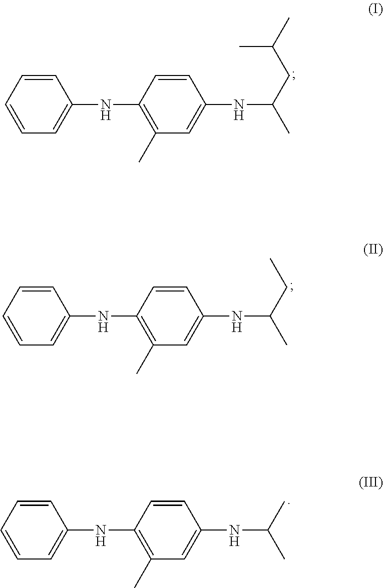 [SMILES: Cc1cc(NC(C)CC(C)C)ccc1Nc1ccccc1 ; CCC(C)Nc1ccc(Nc2ccccc2)c(C)c1 ; Cc1cc(NC(C)C)ccc1Nc1ccccc1]

The present invention further provides a rubber composition, wherein the raw materials of the rubber composition comprise 100 parts by mass of a diene elastomer and 0.5-5 parts by mass of the compound of formula A.

In one or more embodiments of the present invention, the raw materials of the rubber composition comprise 100 parts by mass of a diene elastomer, 0.2-5 parts by mass of a cobalt salt, and 0.5-5 parts by mass of the compound of formula A.

In one or more embodiments of the present invention, the diene elastomer is natural rubber.

In one or more embodiments of the present invention, the raw materials of the rubber composition further comprise 2-10 parts by mass of sulfur.

In one or more embodiments of the present invention, in the raw materials of the rubber composition, the amount of the compound of formula A is 1-3 parts by mass.

In one or more embodiments of the present invention, the raw materials of the rubber composition further comprise 30-70 parts by mass of a reinforcing filler, and preferably, the reinforcing filler comprises carbon black and silica.

In one or more embodiments of the present invention, the raw materials of the rubber composition further comprise 2-15 parts by mass of an activator, and preferably, the activator is zinc oxide.

In one or more embodiments of the present invention, the raw materials of the rubber composition further comprise 0.5-5 parts by mass of a tackifying resin.

In one or more embodiments of the present invention, the raw materials of the rubber composition further comprise 1-10 parts by mass of a rubber adhesive.

In one or more embodiments of the present invention, the raw materials of the rubber composition further comprise 0.5-5 parts by mass of an accelerator, and preferably, the accelerator is N,N′-dicyclohexyl-2-benzothiazolesulfenamide.

In one or more embodiments of the present invention, the raw materials of the rubber composition further comprise 0.02-0.5 parts by mass of a rubber scorch retarder.

The present invention further provides a rubber product comprising the rubber composition according to any embodiments of the present invention. Preferably, the rubber product is a tire belt.

The present invention further provides a method of use of the compound of formula A according to any embodiments of the present invention in improving the mechanical performance and adhesion performance to steel wires after thermal oxidative aging in the presence of metal ion for a rubber composition or rubber product.

In one or more embodiments of the present invention, the method of use comprises adding 0.5-5 parts by mass of the compound of formula A to the raw materials of a rubber composition which comprises 100 parts by mass of a diene elastomer and 0.2-5 parts by mass of a cobalt salt.

### DETAILED DESCRIPTION OF THE INVENTION

In order to enable those skilled in the art to understand the characteristics and effects of the present invention, the terms and expressions mentioned in the description and claims are generally described and defined below. Unless otherwise specified, all technical and scientific terms used in the text have their usual meanings as understood by those skilled in the art regarding the present invention. In the event of conflict, the definitions in this specification shall prevail.

The theories or mechanisms described and disclosed herein, whether true or false, should not limit the scope of the invention in any way, that is, the invention may be implemented without being limited to any specific theory or mechanism.

In the present invention, “comprises”, “comprises”, “contains” and similar terms encompass the meanings of “consisting essentially of” and “consisting of”, for example, when it is disclosed herein that “A comprises B and C”, “A consists essentially of B and C” and “A consists of B and C” shall be deemed to have been disclosed herein.

In the present invention, all characteristics such as numerical values, amounts, contents, and concentrations defined in the form of numerical ranges or percentage ranges are for brevity and convenience only. Accordingly, descriptions of numerical ranges or percentage ranges shall be deemed to cover and specifically disclose all possible subranges and individual values within the ranges (including integers and fractions).

In the present invention, unless otherwise specified, percentage refers to mass percentage, and ratio refers to mass ratio.

In the present invention, when describing embodiments or examples, it should be understood that it is not intended to limit the present invention to these embodiments or examples. Conversely, all substitutes, modifications and equivalents of the methods and materials described in the present invention may be covered within the scope of the claims.

In the present invention, for the sake of conciseness, not all possible combinations of each technical feature in each embodiment or example are described. Thus, as long as there is no contradiction in the combination of these technical features, each technical feature in each embodiment or example may be arbitrarily combined, and all possible combinations should be considered to be within the scope of the specification.

The present invention provides a compound of formula A that is used as a rubber antidegradant:

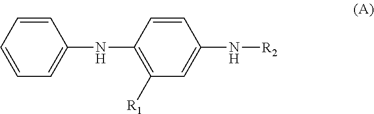

wherein R₁ is selected from C1-C8 alkyl, R₂ is selected from C1-C8 alkyl in formula A.

In the present invention, an alkyl group refers to a monovalent saturated group consisting of hydrogen atoms and carbon atoms. An alkyl group may be a linear or branched alkyl group.

An alkyl group may contain 1 to 8 carbon atoms (C1-C8 alkyl). Examples of an alkyl group include, but are not limited to, methyl, ethyl, isopropyl, n-butyl, isobutyl, 1-methylpropyl, 1,3-dimethylbutyl, 1,4-dimethylpentyl, and octyl, etc.

In some embodiments, in the compound of formula A, R₁ is selected from C1-C₄ alkyl, for example C1-C2 alkyl. In some embodiments, in the compound of formula A, R₁ is methyl.

In some embodiments, in the compound of formula A, R₂ is selected from C₃-C₆ alkyl. In some embodiments, in the compound of formula A, R₂ is selected from isopropyl, 1-methylpropyl, or 1,3-dimethylbutyl.

In some embodiments, in the compound of formula A, R₁ is selected from C₁-C₄ alkyl, for example C1-C2 alkyl; R₂ is selected from C1-C8 alkyl, for example a C₃-C₆ alkyl.

In some embodiments, in the compound of formula A, R₁ is methyl, and R₂ is selected from C₃-C₆ alkyl.

In some embodiments, the compound of formula A is a compound of formula I, a compound of formula II, or a compound of formula III as shown below:

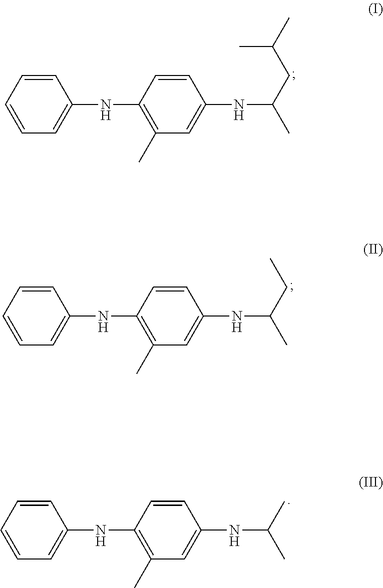 [SMILES: Cc1cc(NC(C)CC(C)C)ccc1Nc1ccccc1 ; CCC(C)Nc1ccc(Nc2ccccc2)c(C)c1 ; Cc1cc(NC(C)C)ccc1Nc1ccccc1]

The present invention provides a method of using the compound A, where adding the compound of formula A (e.g., the compound of formula I, the compound of formula II, or the compound of formula III) to a rubber composition (e.g., a rubber composition for tires, particularly a rubber composition for tire belts) provides excellent thermal oxidative aging resistance and good adhesion performance to steel wire for a rubber composition, especially thermal oxidative aging resistance in the presence of metal ion (e.g., a cobalt salt), significantly improving the mechanical properties and rubber-steel wire adhesion strength after thermal oxidative aging in the presence of metal ion for the rubber composition.

The present invention provides a method for preparing the compound of formula A, which comprises the following steps:

(1) performing a condensation reaction of aniline and a compound of formula B in the presence of a first catalyst to obtain a condensate comprising a compound of formula C and/or a compound of formula C′, and then performing a reduction reaction of the condensate in the presence of hydrogen H₂ and a second catalyst to obtain a compound of formula D:

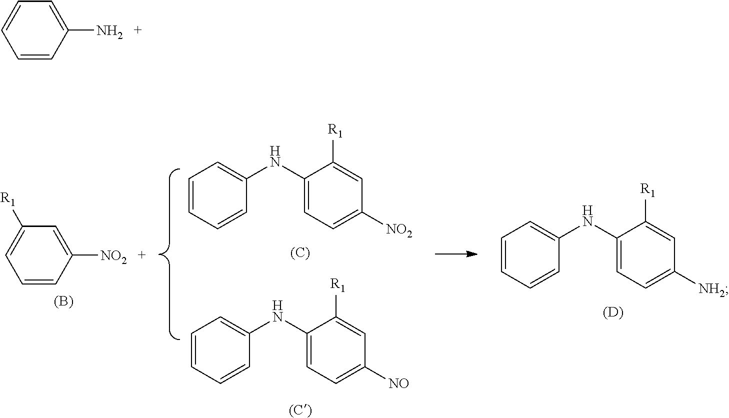

(2) performing a reductive alkylation reaction of the compound of formula D and a compound of formula E in the presence of hydrogen H₂ and a third catalyst to obtain the compound of formula A:

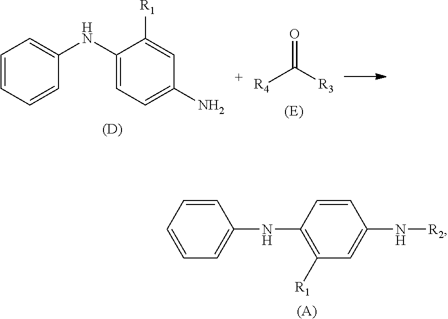

wherein in Formula A, Formula B, Formula C, Formula C′ and Formula D, each R₁ and R₂ are as defined in the embodiments of the present prevention. In Formula E, R₃ and R₄ are each independently selected from H and C1-C7 alkyl. Those skilled in the art can determine the corresponding R₃ and R₄ in Formula E based on R₂ in the compound of Formula A.

The first catalyst used in step (1) may be one or more selected from an alkali metal hydroxide, an alkali metal alkoxide, a quaternary ammonium base, and a combination of an alkali metal hydroxide and a halide of tetraalkylammonium. An alkali metal hydroxide suitable for the present invention comprises sodium hydroxide, potassium hydroxide, lithium hydroxide, etc. An alkali metal alkoxide suitable for the present invention comprises sodium methoxide, potassium methoxide, sodium ethoxide, potassium ethoxide, sodium tert-butoxide, potassium tert-butoxide, sodium tert-pentoxide, potassium tert-pentoxide, etc. A quaternary ammonium base is a compound having the general formula (Rₐ)₄NOH, wherein Rₐ is four identical or different aliphatic hydrocarbon groups (such as alkyl) or aromatic hydrocarbon groups. In some embodiments, each Rₐ in a quaternary ammonium base is an alkyl group, for example, each Rₐ may be independently selected from methyl, ethyl, propyl, and butyl. Examples of a quaternary ammonium base suitable for the present invention include tetraalkylammonium hydroxides, for examples, tetramethylammonium hydroxide, tetraethylammonium hydroxide, and tetrabutylammonium hydroxide. The halide of tetraalkylammonium has the general formula (R_{b})₄NX, wherein R_{b} is four identical or different aliphatic hydrocarbon groups (such as alkyl) or aromatic hydrocarbon groups, such as methyl, ethyl, propyl, butyl, etc., and X is a halogen atom, such as fluorine, chlorine, bromine, and iodine. The first catalyst may also be a combination of an alkali metal hydroxide and a halide of tetraalkylammonium. Examples of the combination of an alkali metal hydroxide and a halide of tetraalkylammonium include sodium hydroxide and tetrabutylammonium bromide. In some embodiments, the first catalyst is a quaternary ammonium base, such as tetraalkylammonium hydroxide. A molar ratio of a first catalyst to aniline may be 0.1:1 to 2:1, preferably 0.1:1 to 0.5:1, such as 0.2:1, 0.3:1, and 0.4:1. In step (1), aniline may firstly react with the first catalyst to form a salt, and then the compound of formula B is added dropwise for a condensation reaction.

In step (1), the condensate is obtained by the condensation reaction of aniline and the compound of formula B in the presence of the first catalyst, and the condensate may be one or two of the compound of formula C and the compound of formula C′, and may also contain an azobenzene compound:

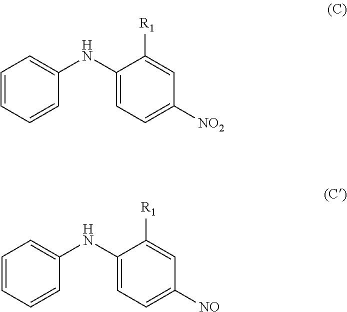

In step (1), the molar ratio of aniline to the compound of formula B may be 2:1˜15:1, preferably 4:1˜10:1, and more preferably 5:1˜8:1, such as 6:1 or 7:1.

In step (1), the condensation reaction may be carried out at 40˜90° C., preferably 65˜85° C., for examples, the reaction temperature may be 60° C., 70° C., 75° C., or 80° C. The condensation reaction needs to be carried out under vacuum with a pressure of −0.09˜−0.99 MPa.

The second catalyst used in step (1) may be a porous metal catalyst or a supported metal catalyst. The porous metal catalyst is also known as a sponge metal catalyst. Porous metal catalysts suitable for the present invention comprise Raney nickel (also known as skeletal nickel), Raney cobalt, and Raney copper, etc. The supported metal catalyst comprises a metal that acts as center of catalytically activity and a support that is used for supporting the metal. The metal in the supported metal catalyst suitable for the present invention may be nickel, cobalt, copper, platinum, palladium, ruthenium, or rhodium, etc., and the support may be carbon, alumina, silica gel, or molecular sieves, etc. The carbon as a support may be activated carbon. In some embodiments, the second catalyst is a porous metal catalyst, such as Raney nickel. The molar ratio of a metal in the second catalyst to a condensate may be 0.0001:1˜0.2:1.

In step (1), the condensate generated by the condensation reaction is subjected to a hydrogenation reduction reaction in the presence of the second catalyst to generate the compound of formula D. In step (1), the reduction reaction may be carried out at 40-120° C., preferably 60˜90° C., for examples, a reaction temperature may be 70° C., 75° C., or 80° C. A hydrogen pressure in the reduction reaction may be 0.5˜5 MPa, preferably 0.5˜2.5 MPa, such as 1 MPa, 1.5 MPa, 2 MPa, or 2.5 MPa.

In step (1), the solvent may be aniline, toluene, or xylene. After the completion of the reaction in step (1), the reaction solution is filtered to recover the second catalyst and the oil-water phase is separated to recover the first catalyst. Then, the organic phase is distilled to remove the light components, thereby obtaining the compound of formula D.

The third catalyst used in step (2) may be the aforementioned supported metal catalyst, such as Pt/C. The molar ratio of the metal in the third catalyst to the compound of formula D may be 0.0001:1˜0.2:1.

In step (2), the compound of formula D and the compound of formula E undergo hydrogenative reductive alkylation reaction in the presence of the third catalyst to generate the compound of formula A. After the reaction, the carbon atom of carbonyl in the compound of formula E is linked to the nitrogen atom of amino in the compound of formula D. Therefore, the compound of formula E may be properly selected for the reaction according to the R₂ group in the compound of formula A to be prepared. The molar ratio of the compound of formula E to the compound of formula D may be 1:1˜15:1, such as 2:1, 3:1, 5:1, 8:1, 10:1, 11:1, or 14:1. A reaction temperature of step (2) may be 40˜150° C., preferably 50˜120° C., such as 50° C., 70° C., 80° C., 100° C., or 120° C. The hydrogen pressure in step (2) may be 0.5˜5 MPa, preferably 0.5˜2.5 MPa, such as 1 MPa, 1.2 MPa, 1.5 MPa, 1.8 MPa, 2 MPa, or 2.5 MPa.

In step (2), the compound of formula E as the raw material for reaction may be used as the solvent. After the completion of the reaction in step (2), the reaction solution is filtered to recover the third catalyst and distilled under reduced pressure to remove the light components, thereby obtaining the compound of formula A.

In the present invention, liquid chromatography (LC) or gas chromatography (GC) may be used to determine whether each step of the reaction reaches the endpoint, thereby determining the appropriate reaction time.

The present invention further provides a rubber composition which comprises the compound of formula A, such as the compound of formula I and/or the compound of formula II.

In the present invention, the term “raw materials” of the rubber composition refers to the formulation of the rubber composition, where various components are mixed together to form the rubber composition, which can be unvulcanized or further vulcanized. The “raw materials” are these different components of the rubber composition before they undergo crosslinking/vulcanization treatment.

The raw materials of the rubber composition typically comprise a diene elastomer, a reinforcing filler, an antidegradant, and a crosslinking agent. In the present invention, the rubber composition comprises an unvulcanized rubber and a vulcanized rubber. The vulcanized rubber may be prepared by vulcanizing (curing) an unvulcanized rubber.

The raw materials of the rubber composition of the present invention comprise a diene elastomer and the compound of formula A as an antidegradant. The amount of the compound of formula A may be 0.5˜5 parts by mass, based on 100 parts by mass of the diene elastomer. In the present invention, unless otherwise specified, the mass of the diene elastomer is taken as 100 parts by mass contained in the raw materials of the rubber composition as the benchmark to calculate the mass parts of other components in the raw materials of the rubber composition.

In the present invention, a diene elastomer refers to an elastomer whose monomer comprises a diene, such as butadiene and isoprene. The diene elastomer suitable for the present invention may be various diene elastomers known in the art, including but not limited to, one or more of natural rubber (NR), butadiene rubber (BR), isoprene rubber, styrene-butadiene rubber (SBR), chloroprene rubber (CR), nitrile rubber (NBR), an isoprene/butadiene copolymer, an isoprene/styrene copolymer, and an isoprene/butadiene/styrene copolymer. In some preferred embodiments, a diene elastomer comprises a natural rubber, or consists of a natural rubber.

The antidegradant contained in the rubber composition of the present invention comprises the compound of formula A (such as the compound of formula I and/or the compound of formula II). In some embodiments, the rubber composition of the present invention does not comprise other antidegradants except for the compound of formula A. In the raw materials of the rubber composition of the present invention, the amount of the compound of formula A may be 0.5-5 parts by mass, preferably 1-3 parts by mass, such as 1.2 parts by mass, 1.5 parts by mass, 1.8 parts by mass, 2 parts by mass, 2.2 parts by mass, or 2.5 parts by mass. In some embodiments, the raw materials of the rubber composition of the present invention comprise the compound of formula I and/or the compound of formula II, preferably comprising the compound of formula I. In some embodiments, the antidegradant in the rubber composition of the present invention is the compound of formula I, the compound of formula II, or the combination of the compound of formula I and the compound of formula II, preferably the compound of formula I. In the raw materials of the rubber composition of the present invention, the amount of the compound of formula I when using a compound of formula I alone, the amount of the compound of formula II when using the compound of formula II alone, the amount of the compound of formula III when using the compound of formula III alone, or the total amount of two or three of the compound of formula I, the compound of formula II, and the compound of formula III when using two or three of the compound of formula I, the compound of formula II, and the compound of formula III at the same time, may be 0.5-5 parts by mass, preferably 1-3 parts by mass, such as 1.2 parts by mass, 1.5 parts by mass, 1.8 parts by mass, 2 parts by mass, 2.2 parts by mass, or 2.5 parts by mass.

The raw materials of the rubber composition of the present invention may comprise a reinforcing filler. In the raw materials of the rubber composition of the present invention, the amount of the reinforcing filler may be 30-70 parts by mass, such as 40 parts by mass, 45 parts by mass, 50 parts by mass, 55 parts by mass, or 60 parts by mass. The reinforcing filler suitable for the present invention may be a reinforcing filler conventionally used in a rubber composition, including but not limited to, one or more selected from the group consisting of carbon black, silica, titanium dioxide, calcium carbonate, magnesium carbonate, aluminum hydroxide, magnesium hydroxide, clay, and talc. In some embodiments, the reinforcing filler comprises carbon black. Examples of carbon black include carbon black N330. In the rubber composition of the present invention, the amount of carbon black may be 25-55 parts by mass, such as 30 parts by mass, 35 parts by mass, 40 parts by mass, 43 parts by mass, 45 parts by mass, or 50 parts by mass. In some embodiments, the reinforcing filler comprises silica. In the rubber composition of the present invention, the amount of silica may be 5-20 parts by mass, such as 8 parts by mass, 10 parts by mass, 12 parts by mass, or 15 parts by mass. In some preferred embodiments, the reinforcing filler comprises carbon black and silica, or consists of carbon black and silica.

The raw materials of the rubber composition of the present invention comprise a crosslinking agent, such as sulfur. Sulfur is preferably an insoluble sulfur. In the raw materials of the rubber composition of the present invention, the amount of sulfur may be 2-10 parts by mass, such as 3 parts by mass, 4 parts by mass, 5 parts by mass, 6 parts by mass, 7 parts by mass, 8 parts by mass, or 9 parts by mass.

The raw materials of the rubber composition of the present invention may also comprise other components commonly used in a rubber composition, including but not limited to, one or more selected from the group consisting of a softener, a protective wax, an activator, a cobalt salt, a tackifying resin, a rubber adhesive, an accelerator, a rubber scorch retarder, and the like.

A softener may be used to improve the processability of the rubber composition. Softener may comprise a petroleum-based softener such as naphthenic oil, aromatic oil, processing oil, lubricating oil, paraffin, liquid paraffin, petroleum bitumen, and petrolatum, etc., and may also comprise a fatty oil-based softener such as stearic acid, castor oil, linseed oil, rapeseed oil, coconut oil, waxes (such as beeswax, carnauba wax, and lanolin), tall oil, linoleic acid, palmitic acid, and lauric acid, etc. In the present invention, a softener is optionally added. When the raw materials of the rubber composition of the present invention comprise a softener, the amount of the softener may be 1-10 parts by mass, preferably 3-9 parts by mass, such as 4 parts by mass, 5 parts by mass, 6 parts by mass, 7 parts by mass, or 8 parts by mass.

A protective wax may migrate from the interior of a rubber to the surface to form a wax film, isolating the rubber surface from the external environment. In the present invention, a protective wax is optionally added. When the raw materials of the rubber composition of the present invention comprise a protective wax, the amount of the protective wax may be 1-5 parts by mass, such as 1.5 parts by mass, 2 parts by mass, 3 parts by mass, or 4 parts by mass.

An activator may play a role in accelerating the vulcanization rate, improving the thermal conductivity, wear resistance, and tear resistance of a rubber. The raw materials of the rubber composition of the present invention preferably comprise an activator. Examples of an activator include ZnO. In the raw materials of the rubber composition of the present invention, the amount of activator may be 2-15 parts by mass, such as 5 parts by mass, 6 parts by mass, 7 parts by mass, 8 parts by mass, 9 parts by mass, 10 parts by mass, or 12 parts by mass. In some preferred embodiments, the activator comprises ZnO, or the activator is ZnO. In some preferred embodiments, in the raw materials of the rubber composition of the present invention, the amount of ZnO is 2-15 parts by mass, such as 5 parts by mass, 6 parts by mass, 7 parts by mass, 8 parts by mass, 9 parts by mass, 10 parts by mass, or 12 parts by mass.

A cobalt salt may greatly help to promote rubber-steel wire adhesion. When used in a rubber composition for tire belt, the raw materials of the rubber composition of the present invention preferably comprise a cobalt salt. Cobalt salts that can be used in the present invention comprise, but are not limited to, cobalt borate and cobalt neodecanoate. In the raw materials of the rubber composition of the present invention, the amount of a cobalt salt may be 0.2˜5 parts by mass, such as 0.5 parts by mass, 0.8 parts by mass, 1 part by mass, 1.2 parts by mass, 1.5 parts by mass, 2 parts by mass, or 3 parts by mass.

A tackifying resin is used to enhance the adhesion of rubber to steel cord. When used in a rubber composition for tire belt, the raw material of the rubber composition of the present invention preferably comprises a tackifying resin. A usable tackifying resin comprises a resorcinol donor, such as resorcinol-formaldehyde resin. In the raw material of the rubber composition of the present invention, the amount of a tackifying resin may be 0.5-5 parts by mass, such as 1 part by mass, 1.5 parts by mass, 2 parts by mass, 2.5 parts by mass, 3 parts by mass, and 4 parts by mass. In some embodiments, the tackifying resin comprises resorcinol-formaldehyde resin, or the tackifying resin is resorcinol-formaldehyde resin. Examples of usable tackifying resins include resorcinol-formaldehyde resin SL3022.

A rubber adhesive may be an adhesive containing a methylene donor (such as hexamethoxymethylmelamine), which is used in combination with a tackifying resin to fully adhere the rubber to a skeleton material such as a steel wire. When using as a rubber composition for tire belt, the raw materials of the rubber composition of the present invention preferably comprise a rubber adhesive. An available rubber adhesive comprises a rubber adhesive with an active ingredient of hexamethoxymethylmelamine (HMMM), such as rubber adhesive RA. Rubber adhesive RA consists of HMMM and an inorganic carrier. Examples of rubber adhesive RA include rubber adhesive RA-65. The mass fraction of HMMM in rubber adhesive RA-65 is 65%. In the raw materials of the rubber composition of the present invention, the amount of rubber adhesive may be 1-10 parts by mass, such as 2 parts by mass, 3 parts by mass, 4 parts by mass, 5 parts by mass, 6 parts by mass, 7 parts by mass, or 8 parts by mass. In some embodiments, a rubber adhesive comprises rubber adhesive RA, or a rubber adhesive is rubber adhesive RA. In the raw materials of the rubber composition of the present invention, the amount of methylene donor (such as HMMM) may be 1-10 parts by mass, such as 2 parts by mass, 3 parts by mass, 4 parts by mass, 5 parts by mass, 6 parts by mass, 7 parts by mass, or 8 parts by mass.

A rubber scorch retarder is used to prevent the vulcanization of the rubber composition in the early stage of processing, and a rubber scorch retarder may be one or more selected from a nitroso compound (such as N-nitrosodiphenylamine), an organic acid (such as benzoic acid, phthalic anhydride), and a thioamide (such as N-cyclohexylthiophthalimide). Examples of rubber scorch retarders include rubber scorch retarder CTP (N-cyclohexylthiophthalimide). The raw materials of the rubber composition of the present invention preferably comprise a rubber scorch retarder. In the raw materials of the rubber composition of the present invention, an amount of a rubber scorch retarder may be 0.02-0.5 parts by mass, such as 0.05 parts by mass, 0.1 parts by mass, 0.2 parts by mass, 0.3 parts by mass, or 0.4 parts by mass. In some embodiments, a rubber scorch retarder comprises rubber scorch retarder CTP, or a rubber scorch retarder is rubber scorch retarder CTP.

An accelerator is usually a vulcanization accelerator, and may be one or more of a sulfonamide vulcanization accelerator, a thiazole vulcanization accelerator, a thiuram vulcanization accelerator, a thiourea vulcanization accelerator, a guanidine vulcanization accelerator, a dithiocarbamate vulcanization accelerator, a aldehyde amine vulcanization accelerator, a aldehyde ammonia vulcanization accelerator, an imidazoline vulcanization accelerator, and a xanthonic acid vulcanization accelerator. In the raw materials of the rubber composition of the present invention, the amount of the accelerator may be 0.5-5 parts by mass, such as 1 part by mass, 1.4 parts by mass, 1.5 parts by mass, 2 parts by mass, or 3 parts by mass. In some preferred embodiments, an accelerator is accelerator DZ (N,N′-dicyclohexyl-2-benzothiazolylsulfenamide).

In addition, if it is required, the rubber composition may further comprise a plasticizer such as DMP (dimethyl phthalate), DEP (diethyl phthalate), DBP (dibutyl phthalate), DHP (diheptyl phthalate), DOP (dioctyl phthalate), DINP (diisononyl phthalate), DIDP (diisodecyl phthalate), BBP (butyl benzyl phthalate), DWP (dilauryl phthalate), and DCHP (dicyclohexyl phthalate). The amount of a plasticizer may be a routine amount known in the art.

When the rubber composition is used as a rubber composition for the tire belt, the rubber composition of the present invention preferably comprises a bonding system consisting of a resorcinol donor (such as resorcinol-formaldehyde resin), a methylene donor (such as MMM), and silica, which may effectively improve the adhesion of rubber to metal. The amount of the resorcinol donor, the methylene donor, and silica can be as described in any embodiments of the present invention.

In some preferred embodiments, the raw materials of the rubber composition of the present invention comprise: 100 parts by mass of a diene elastomer, 2-10 parts by mass of sulfur, 0.5-5 parts by mass of the compound of formula A, 25-55 parts by mass of carbon black, 5-20 parts by mass of silica, 2-15 parts by mass of ZnO, 0.2-5 parts by mass of a cobalt salt, 0.5-5 parts by mass of a tackifying resin (such as resorcinol-formaldehyde resin), 1-10 parts by mass of a rubber adhesive (such as rubber adhesive RA), 0.02-0.5 parts by mass of a rubber scorch retarder (such as rubber scorch retarder CTP), and 0.5-5 parts by mass of an accelerator (such as accelerator DZ), or consist of the above components.

An unvulcanized rubber of the present invention may be prepared by a conventional rubber mixing method, for example, it may be prepared by a two-stage mixing method: a first stage of thermomechanical mixing (e.g., using a internal mixer), mixing the raw materials of the rubber composition other than a crosslinking agent, a rubber adhesive, and an accelerator to obtain a mixture, and then kneading the mixture until reaching a maximum temperature between 110° C. and 190° C. to obtain a first stage rubber; a second stage of thermomechanical mixing (e.g., using an open mill), mixing the first stage rubber with a crosslinking agent, a rubber adhesive, and an accelerator to obtain a second stage rubber, i.e., an unvulcanized rubber.

An unvulcanized rubber of the present invention may be vulcanized by a conventional vulcanization method to obtain a vulcanized rubber. The vulcanization temperature is usually 130° C.-200° C., such as 140-160° C. or 150±5° C.; the vulcanization time depends on the vulcanization temperature, vulcanization system, and vulcanization kinetics, and is usually 15-60 minutes, such as 20-40 minutes or 30±5 minutes.

Compared with a rubber composition using antidegradant 6PPD, the rubber composition of the present invention, which uses the compound of formula A (such as one or more of the compound of formula I, the compound of formula II, and the compound of formula III) as an antidegradant, is used in a rubber product such as a tire, especially a tire belt, so that the rubber product has better mechanical properties and rubber-steel wire adhesion performance after thermal-oxidative aging in the presence of metal ion (such as cobalt ion). Therefore, the present invention also provides a rubber product which comprises the rubber composition described in the present invention. A rubber product may be a tire, a rubber shoe, a sealing strip, an acoustic panel, or a crash pad, etc. Preferably, a rubber product is a tire, such as a tire belt.

The present invention further provides a method of using the compound of Formula A (e.g., one or more of the compound of formula I, the compound of formula II, and the compound of formula III) in improving the mechanical performance and rubber-steel wire adhesion performance after thermo-oxidative aging in the presence of metal ion (e.g., cobalt ion) for the rubber composition or rubber product, and also provides a method for improving the mechanical performance and rubber-steel wire adhesion performance after thermo-oxidative aging in the presence of metal ion (e.g., cobalt ion) for the rubber composition or rubber product. Preferably, the method of use of the present invention comprises: adding the compound of formula A (e.g., one or more of the compound of formula I, the compound of formula II, and the compound of formula III) to the raw materials of a rubber composition. In the method of the present invention, the amount of the compound of formula A (e.g., one or more of the compound of formula I, the compound of formula II, and the compound of formula III) and the composition of the raw materials of the rubber composition are preferably as described in any embodiments of the present invention.

The present invention is described in the following examples. These examples are merely illustrative and are not intended to limit the scope of the present invention. The methods, reagents, and materials used in the examples, unless otherwise stated, are conventional methods, reagents and materials in the art. The raw materials used in the examples are commercially available.

The sources and specifications of the raw materials used in the examples are as follows:
Natural rubber (NR): Xishuangbanna Sinochem Rubber Co., Ltd., SCR5;
Carbon black: Shanghai Cabot Carbon Black Co., Ltd., N330;
Silica: Shanghai Cain Chemical Co., Ltd., VN3;
Zinc oxide: Yonghua Chemical Technology (Jiangsu) Co., Ltd.;
Cobalt borate: Zhejiang Jinmao Rubber Auxiliaries Products Co., Ltd., compliant with industry standard HG/T 4072-2008;
Antidegradant 6PPD: Sennics Co., Ltd.;
Resorcinol-formaldehyde resin: SI Group Co., Ltd., SL3022;
Rubber adhesive RA-65: Shandong Ruiqi Chemical Co., Ltd.;
Rubber scorch retarder CTP: Shandong Yanggu Huatai Chemical Co., Ltd.;
Insoluble sulfur: Sennics Co., Ltd., HD OT20;
Accelerator DZ: Willing New Materials Technology Co., Ltd.

### Preparation Example 1

### (1) Synthesis of Compound IV

139.7 g (1.5 mol) of aniline and 91 g (0.25 mol) of 25% aqueous solution of tetramethylammonium hydroxide (TMAOH) are added into a 500 mL four-mouth flask with stirring and the temperature is raised to 40˜55° C. TMAOH and aniline are formed into salts by distillation and dehydration under reduced pressure. During the process, the color of the reaction liquid gradually changes from yellow to dark-red. The temperature is gradually raised to 72° C. When the amount of fraction is about 46 mL, distillation under reduced pressure(−0.098 MPa) at 72° C. and dropwise addition of 34.3 g (0.25 mol) 3-methyl nitrobenzene are performed at the same time, and the dropwise addition time is about 3 hrs. After the dropwise addition, the temperature is kept for 1 hr. The reaction is monitored by LC until 3-methyl nitrobenzene is completely reacted. A condensate liquid is obtained.

The above condensate liquid is transferred to a 500 mL stainless steel reactor, to which 50 g of deionized water and 40 g of skeleton nickel catalyst are added. The atmosphere is replaced with hydrogen for three times, the temperature is raised to 75° C. and hydrogen is introduced to raise the pressure to be 1.5 MPa for a hydro-reduction reaction. The reaction is monitored by LC until nitro-compounds and nitroso-compounds are completely reduced. Then the reaction liquid is filtered and separated to obtain an organic phase, and the organic phase is washed with water and distilled under reduced pressure (−0.1 MPa, 160° C.) to remove aniline and by-products of light components. Finally, 42.6 g of a reduction product (Compound IV) is obtained through rectification, and it is cooled and solidified into a yellow solid. The yield of Compound IV is about 86% and its content is >99% by GC detection.

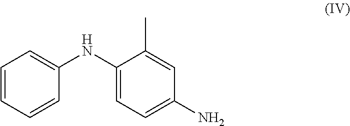

LC-MS (m/z): 198.22 (M-H+).

1H NMR (400 MHz, CDCl3) δ 7.22-7.15 (m, 2H), 7.03 (d, J=8.3 Hz, 1H), 6.79-6.74 (m, 1H), 6.68 (dt, J=8.8, 1.7 Hz, 2H), 6.61 (d, J=2.6 Hz, 1H), 6.54 (dd, J=8.3, 2.7 Hz, 1H), 5.15 (s, 1H), 3.56 (s, 2H), 2.17 (s, 3H).

### (2) Synthesis of Compound I

46 g (0.23 mol) of Compound IV, 70 g (0.70 mol) of 4-methyl-2-pentanone, and 0.8 g of Pt/C catalyst are put into a reactor. The atmosphere is replaced with hydrogen for three times. The temperature is raised to 80° C., and hydrogen is introduced to raise the pressure to be 1.8 MPa for a reaction. The reaction is monitored by GC. When the content of Compound IV is <0.1% by GC detection, the reaction is stopped. The temperature is cooled down, the catalyst is removed by filtration, and the light components are removed by distillation under a pressure of −0.1 MPa at 180° C. to obtain 60.4 g of Compound I (yield is about 93%), and its content is >95.8% by GC detection. After cooling and solidification, it is a red liquid.

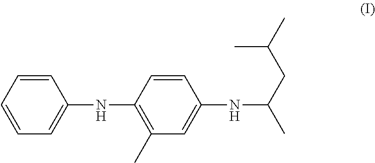 [SMILES: Cc1cc(NC(C)CC(C)C)ccc1Nc1ccccc1]

Molecular formula: C₁₉H₂₆N₂

LC-MS (m/z): 282.40 (M-H+).

### Preparation Example 2

### (1) Synthesis of Compound IV

The synthesis of Compound IV is the same as Preparation Example 1.

### (2) Synthesis of Compound II

40 g (0.2 mol) of Compound IV, 151.4 g (2.1 mol) of 2-butanone, and 0.5 g of Pt/C catalyst are added into a reactor. The atmosphere is replaced with hydrogen for three times.

The temperature is raised to 80° C., and hydrogen is introduced to raise the pressure to be 1.5 MPa for a reaction. The reaction is monitored by GC, when the content of Compound IV is <0.1%, the reaction is stopped. The temperature is cooled down. The catalyst is removed by filtration, and light components are removed by distillation under a pressure of −0.1 MPa at 180° C. to obtain 50.9 g of Compound II (yield is about 99%), and its content is >99% by GC detection. After cooling and solidification, it is a light red solid.

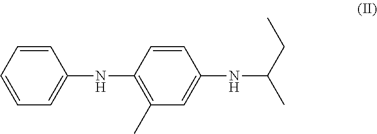 [SMILES: CCC(C)Nc1ccc(Nc2ccccc2)c(C)c1]

Molecular formula: C₁₇H2₂N2

LC-MS(m/z): 254.35 (M-H+).

¹H NMR (400 MHz, CDCl₃) δ 7.26-7.18 (m, 2H), 7.01 (d, J=8.4 Hz, 1H), 6.73-6.68 (m, 1H), 6.65 (m, 2H), 6.47 (d, J=2.7 Hz, 1H), 6.43 (dd, J=8.4, 2.7 Hz, 1H), 4.98 (s, 1H), 3.68-3.42 (m, 1H), 3.30 (br.s, 1H), 2.20 (s, 3H), 1.52-1.45 (m, 2H), 1.21 (d, J=6.3 Hz, 3H), 0.97 (t, J=7.4 Hz, 3H).

### Preparation Example 3

### (1) Synthesis of Compound IV

The synthesis of Compound IV is the same as Preparation Example 1.

### (2) Synthesis of Compound III

40 g (0.2 mol) of Compound IV, 162.4 g (2.8 mol) of acetone, and 0.5 g of Pt/C catalyst are added into a reactor. The atmosphere is replaced with hydrogen for three times. The temperature is raised to 70° C., and hydrogen is introduced to raise the pressure to be 1.5 MPa for a reaction. The reaction is monitored by GC, when the content of Compound IV is <0.1%, the reaction is stopped. The temperature is cooled down. The catalyst is removed by filtration, and light components are removed by distillation under a pressure of −0.1 MPa at 180° C. to obtain 48.3 g of Compound III (yield is about 99.4%), with a content of >98% by GC detection.

After cooling and solidification, it is a light pink solid.

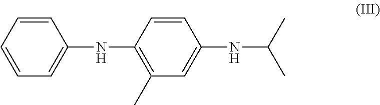 [SMILES: Cc1cc(NC(C)C)ccc1Nc1ccccc1]

Molecular formula: C₁₆H2₀N2

LC-MS (m/z): 240.35 (M-H+).

¹H NMR (400 MHz, CDCl₃) δ 7.20-7.11 (m, 2H), 7.03 (d, J=8.4 Hz, 1H), 6.77-6.70 (m, 1H), 6.65 (dt, J=8.8, 1.7 Hz, 2H), 6.50 (d, J=2.7 Hz, 1H), 6.44 (dd, J=8.4, 2.7 Hz, 1H), 5.13 (s, 1H), 3.71-3.43 (m, 1H), 3.34 (br s, 1H), 2.17 (s, 3H), 1.23 (d, J=6.3 Hz, 6H).

### Examples 1-6 and Comparative Examples 1-4

According to the formulations shown in Table 1, the rubber composition of Examples 1-6 and Comparative Examples 1-4 are prepared using the following steps:

(1) A first-stage mixing is carried out in an internal mixer. The initial temperature of the internal mixer is 70° C., and the rotor speed is 60 r·min⁻¹. Natural rubber is added into the internal mixer, followed by the addition of carbon black N330, silica, zinc oxide, cobalt borate, antidegradant (a compound of Formula I, a compound of Formula II, or antidegradant 6PPD), resorcinol-formaldehyde resin SL3022, and rubber scorch retarder CTP. The mixture is kneaded until reaching a temperature of 150° C. and then discharged to obtain a first-stage rubber.

(2) A second-stage mixing is carried out on an open mill. The first-stage rubber, rubber adhesive RA-65, insoluble sulfur, and accelerator DZ are added into the open mill. After the mixture was sheeted off after 5 passes, a second-stage rubber is obtained.

(3) The second-stage rubber is vulcanized at 151° C. for 30 minutes to obtain a vulcanized rubber.

TABLE 1
Formulations of the rubber composition of Examples 1-6 and Comparative Examples 1-4 (unit: parts by mass)

| | Ex. 1 | Ex. 2 | Ex. 3 | Ex. 4 | Ex. 5 | Ex. 6 | Com. Ex. 1 | Com. Ex. 2 | Com. Ex. 3 | Com. Ex. 4 |
|---|---|---|---|---|---|---|---|---|---|---|
| Natural rubber | 100 | 100 | 100 | 100 | 100 | 100 | 100 | 100 | 100 | 100 |
| Carbon black | 43 | 43 | 43 | 43 | 43 | 43 | 43 | 43 | 43 | 43 |
| N330 | | | | | | | | | | |
| Silica | 10 | 10 | 10 | 10 | 10 | 10 | 10 | 10 | 10 | 10 |
| Zinc oxide | 8 | 8 | 8 | 8 | 8 | 8 | 8 | 8 | 8 | 8 |
| Cobalt borate | 1 | 1 | 1 | 1 | 1 | 1 | 1 | 0 | 1 | 1 |
| Compound I | 2 | 1.5 | 0 | 0 | 0 | 0 | 0 | 0 | 0 | 0 |
| Compound II | 0 | 0 | 2 | 1.5 | 0 | 0 | 0 | 0 | 0 | 0 |
| Compound III | 0 | 0 | 0 | 0 | 2 | 1.5 | 0 | 0 | 0 | 0 |
| Antidegradant | 0 | 0 | 0 | 0 | 0 | 0 | 0 | 0 | 2 | 1.5 |
| 6PPD | | | | | | | | | | |
| Resorcinol- | 2 | 2 | 2 | 2 | 2 | 2 | 2 | 2 | 2 | 2 |
| formaldehyde | | | | | | | | | | |
| resin SL3022 | | | | | | | | | | |
| Insoluble sulfur | 5 | 5 | 5 | 5 | 5 | 5 | 5 | 5 | 5 | 5 |
| Rubber | 5 | 5 | 5 | 5 | 5 | 5 | 5 | 5 | 5 | 5 |
| adhesive RA- | | | | | | | | | | |
| 65 | | | | | | | | | | |
| Accelerator DZ | 1.4 | 1.4 | 1.4 | 1.4 | 1.4 | 1.4 | 1.4 | 1.4 | 1.4 | 1.4 |
| Rubber scorch | 0.1 | 0.1 | 0.1 | 0.1 | 0.1 | 0.1 | 0.1 | 0.1 | 0.1 | 0.1 |
| retarder CTP | | | | | | | | | | |

### Test Example 1: Mechanical Properties Before and After Thermal Oxidative Aging

A tensile strength and an elongation at break before thermal oxidative aging, and a retention rate of tensile strength and a retention rate of elongation at break after thermal oxidative aging, of the vulcanized rubbers are tested according to the following standards:

Tensile strength and elongation at break: GB/T 528-2009, Determination of tensile stress-strain properties of a vulcanized rubber or thermoplastic rubber.

Thermal oxidative aging test: GB/T 3512-2014, Vulcanized or thermoplastic rubber, Hot-air-accelerated aging and heat resistance test, Aging condition of 100° C.×48 h.

Retention rate of tensile strength after aging=(Tensile strength after aging/Tensile strength before aging)×100%

Retention rate of elongation at break after aging=(Elongation at break after aging/Elongation at break before aging)×100%

The mechanical properties before and after aging for the rubber composition of Examples 1-6 and Comparative Examples 1-4 are shown in Table 2.

TABLE 2
Mechanical properties before and after aging for rubber
composition of Examples 1-6 and Comparative Examples 1-4

| | Ex. 1 | Ex. 2 | Ex. 3 | Ex. 4 | Ex. 5 | Ex. 6 | Com. Ex. 1 | Com. Ex. 2 | Com. Ex. 3 | Com. Ex. 4 |
|---|---|---|---|---|---|---|---|---|---|---|
| Tensile | 24.5 | 24.3 | 24.4 | 24.2 | 24.0 | 23.9 | 24.2 | 24.4 | 24.3 | 24.2 |
| strength | | | | | | | | | | |
| before | | | | | | | | | | |
| aging/MPa | | | | | | | | | | |
| Elongation at | 437 | 431 | 435 | 432 | 433 | 430 | 426 | 450 | 438 | 429 |
| break before | | | | | | | | | | |
| aging/% | | | | | | | | | | |
| Retention rate | 69 | 68.2 | 65.8 | 65.1 | 63.7 | 63.0 | 52.1 | 56.2 | 62.5 | 61.9 |
| of tensile | | | | | | | | | | |
| strength after | | | | | | | | | | |
| aging under | | | | | | | | | | |
| 100° C. × | | | | | | | | | | |
| 48 h/% | | | | | | | | | | |
| Retention rate | 47.2 | 46.3 | 45.2 | 44.9 | 43.5 | 43.0 | 32.3 | 36.1 | 41.5 | 41 |
| of elongation | | | | | | | | | | |
| at break after | | | | | | | | | | |
| aging under | | | | | | | | | | |
| 100° C. × | | | | | | | | | | |
| 48 h/% | | | | | | | | | | |

As can be seen from Table 2, the mechanical properties before aging of the rubber composition in Examples 1-2 containing the compound of formula I, Examples 3-4 containing the compound of formula II, and Examples 5-6 containing the compound of formula III are close to those of the rubber composition in Comparative Examples 3-4 containing antidegradant 6PPD. The retention rate of tensile strength and the retention rate of elongation at break after aging of the rubber composition of Comparative Example 1 are lower than those of Comparative Example 2, indicating that the addition of cobalt salt is not conducive to thermal oxidative aging performance. Compared to the rubber composition of Comparative Example 1, the retention rate of tensile strength and the retention rate of elongation at break after aging of the rubber composition in Examples 1-2 containing Compound I, Examples 3-4 containing Compound II, and Examples 5-6 containing Compound III are significantly improved, indicating that Compound I, Compound II, and Compound III can provide excellent thermal oxidative aging resistance and protection against metal ions for a rubber composition. Especially at the same dosage of an antidegradant, the retention rate of tensile strength and the retention rate of elongation at break after thermal oxidative aging in the presence of metal ion for the rubber composition in Examples 1-2 containing Compound I, Examples 3-4 containing Compound II, and Examples 5-6 containing Compound III are higher than those of the rubber composition in Comparative Examples 3-4 containing antidegradant 6PPD, indicating that Compound I, Compound II, and Compound III have better effects in improving the thermal oxidative aging resistance and protection against metal ion of a rubber composition compared to antidegradant 6PPD.

### Test Example 2: Adhesion Performance to Steel Wire

An adhesion strength to steel cord before and after thermal oxidative aging for the vulcanized rubber of Examples 1-6 and Comparative Examples 1-4 is tested according to the following standards, and test results are shown in Table 3:

Adhesion strength: GB/T 16586-1996, Determination of adhesion strength between vulcanized rubber and steel cord.

Thermal oxidative aging test: GB/T 3512-2014, Vulcanized or thermoplastic rubber, Hot-air-accelerated aging and heat resistance tests, Aging condition of 100° C. x 96 h.

Retention rate of adhesion strength after aging=(Adhesion strength after aging/Adhesion strength before aging)×100%

TABLE 3
Adhesion strength to steel cord before and after aging for rubber
composition of Examples 1-6 and Comparative Examples 1-4

| | | | | | | | Com. | Com. | Com. | Com. |
|---|---|---|---|---|---|---|---|---|---|---|
| | Ex. 1 | Ex. 2 | Ex. 3 | Ex. 4 | Ex. 5 | Ex. 6 | Ex. 1 | Ex. 2 | Ex. 3 | Ex. 4 |
| Adhesion strength before | 77 | 76 | 76 | 76 | 75 | 74 | 72 | 68 | 74 | 74 |
| aging/(N/mm) | | | | | | | | | | |
| Retention rate of adhesion | 92.7 | 91.4 | 89.4 | 88.7 | 86.1 | 85.5 | 78.5 | 77.6 | 85.3 | 84.5 |
| strength after aging under | | | | | | | | | | |
| 100° C. × 96 h/% | | | | | | | | | | |

As can be seen from Table 3, compared to the vulcanized rubber of Comparative Example 2 without cobalt salt, the vulcanized rubber of Comparative Example 1 with cobalt salt has higher adhesion strength, indicating that the addition of cobalt salt improves rubber-steel wire adhesion performance. The adhesion strength of the vulcanized rubber in Examples 1-2 containing Compound I and Examples 3-4 containing Compound II is higher than that of the vulcanized rubber in Comparative Example 1 without antidegradant and Comparative Examples 3-4 containing antidegradant 6PPD, indicating that Compound I and Compound II can improve rubber-steel wire adhesion performance. The retention rates of adhesion strength after thermal oxidative aging of the vulcanized rubber in Examples 1-2 containing Compound I, Examples 3-4 containing Compound II, and Examples 5-6 containing Compound III are higher than those of the vulcanized rubber in Comparative Examples 3-4 containing antidegradant 6PPD, indicating that Compound I, Compound II, and Compound III can improve the rubber-steel wire adhesion performance after thermal oxidative aging in the presence of metal ion.

We claim:

1. A rubber composition, wherein a formulation of the rubber composition comprises:
100 parts by mass of a diene elastomer,
0.2-5 parts by mass of a cobalt salt, and
0.5-5 parts by mass of a compound of formula A: 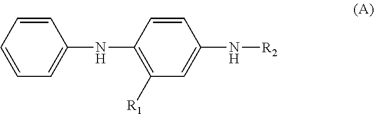
wherein R₁ is a C1-C8 alkyl, and R₂ is a C1-C8 alkyl.

2. The rubber composition according to claim 1, wherein R₁ is methyl, and R₂ is a C3-C6 alkyl.

3. The rubber composition according to claim 1, wherein the compound of formula A is a compound of formula I, a compound of formula II, or a compound of formula III:

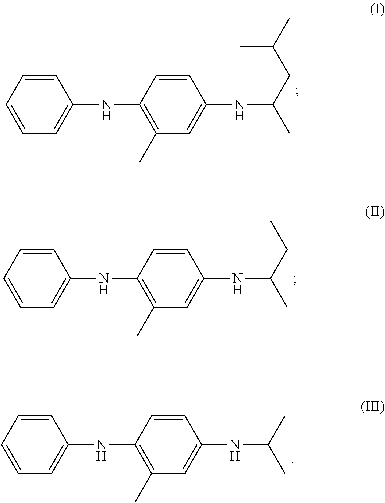 [SMILES: Cc1cc(NC(C)CC(C)C)ccc1Nc1ccccc1 ; CCC(C)Nc1ccc(Nc2ccccc2)c(C)c1 ; Cc1cc(NC(C)C)ccc1Nc1ccccc1]

4. The rubber composition according to claim 1, wherein the diene elastomer is natural rubber.

5. The rubber composition according to claim 1, wherein the formulation of the rubber composition further comprises 2-10 parts by mass of sulfur.

6. The rubber composition according to claim 1, wherein an amount of the compound of formula A is 1-3 parts by mass.

7. The rubber composition according to claim 1, wherein the formulation of the rubber composition further comprises 30-70 parts by mass of a reinforcing filler.

8. The rubber composition according to claim 7, wherein the reinforcing filler comprises carbon black, and silica.

9. The rubber composition according to claim 1, wherein the formulation of the rubber composition further comprises: 2-15 parts by mass of an activator.

10. The rubber composition according to claim 9, wherein the activator is zinc oxide.

11. The rubber composition according to claim 1, wherein the formulation of the rubber composition further comprises 0.5-5 parts by mass of a tackifying resin.

12. The rubber composition according to claim 1, wherein the formulation of the rubber composition further comprises 1-10 parts by mass of a rubber adhesive.

13. The rubber composition according to claim 1, wherein the formulation of the rubber composition further comprises 0.5-5 parts by mass of an accelerator.

14. The rubber composition according to claim 1, wherein the accelerator is N,N′-dicyclohexyl-2-benzothiazolesulfenamide.

15. The rubber composition according to claim 1, wherein the formulation of the rubber composition further comprises 0.02-0.5 parts by mass of a rubber scorch retarder.

16. A rubber product, wherein the rubber product comprises the rubber composition according to claim 1.

17. A method of improving mechanical performance and adhesion performance to steel wire after thermal oxidative aging in presence of metal ion for a rubber composition, comprising:
applying a compound of formula A in a rubber composition, wherein in formula A, 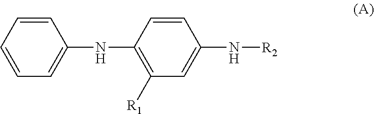
R₁ is a C1-C8 alkyl, and R₂ is a C1-C8 alkyl.

18. The method of claim 17, wherein R₁ is methyl, and R₂ is a C3-C6 alkyl in the compound of formula A.

19. The method of claim 17, wherein the compound of formula A is a compound of formula I, a compound of formula II, or a compound of formula III:

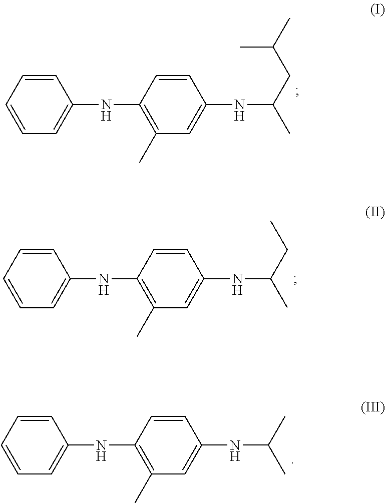 [SMILES: Cc1cc(NC(C)CC(C)C)ccc1Nc1ccccc1 ; CCC(C)Nc1ccc(Nc2ccccc2)c(C)c1 ; Cc1cc(NC(C)C)ccc1Nc1ccccc1]

20. The method of claim 17, further comprising:
adding 0.5-5 parts by mass of the compound of formula A to the rubber composition,
wherein a formulation of the rubber composition comprise 100 parts by mass of a diene elastomer and 0.2-5 parts by mass of a cobalt salt.
